(19) United States
(12) Patent Application Publication
(10) Pub. No.: US 2026/0131049 A1
(43) Pub. Date: May 14, 2026

(54) RESIN COMPOSITION, MEDICAL MATERIAL, AND METHOD FOR PRODUCING RESIN COMPOSITION

(71) Applicant: National University Corporation Tokyo University of Agriculture and Technology, Tokyo (JP)

(72) Inventors: Yasumoto NAKAZAWA, Kanagawa (JP); Shota AKIOKA, Tokyo (JP); Yuri MATSUMOTO, Tokyo (JP)

(21) Appl. No.: 19/401,386

(22) Filed: Nov. 26, 2025

Related U.S. Application Data
(63) Continuation of application No. PCT/JP2024/019851, filed on May 30, 2024.

(30) Foreign Application Priority Data
May 31, 2023 (JP) 2023-089790

(51) Int. Cl. A61L 27/22; A61K 47/34; A61K 47/42; A61L 27/18

(52) U.S. Cl. CPC A61L 27/22; A61K 47/34; A61K 47/42; A61L 27/18

(57) ABSTRACT

The resin composition including the first polymer compound and second polymer compound is provided. The first polymer compound has, in a main chain, a first repeating unit formed of a plurality of amino acid residues bonded by peptide bond. The second polymer compound has, in a terminal of a side chain, a peptide chain or polypeptide chain formed of a plurality of amino acid residues bonded by peptide bond. The plurality of amino acid residues constituting the first repeating unit of the first polymer compound include amino acid residues derived from at least two types of amino acids selected from a group consisting of glycine, alanine, serine, tyrosine, and valine. The plurality of amino acid residues constituting the peptide chain or polypeptide chain of the second polymer compound include the amino acid residues derived from the at least two types of amino acids constituting the first repeating unit.

The contents of the following Japanese patent applications are incorporated herein by reference:
NO. 2023-089790 filed in JP on May 31, 2023
NO. PCT/JP2024/019851 filed in WO on May 30, 2024

### BACKGROUND

### 1. Technical Field

The present invention relates to a resin composition, a medical material, and a method for producing a resin composition.

### 2. Related Art

Patent document 1 discloses a porous material that has a porous part and a coating part containing silk fibroin and alcohol. Patent document 2 discloses a polyurethane resin composition-containing regenerated silk fibroin fiber in which excellent strength and flexibility are consistent. Patent document 3 discloses a silk fibroin biocompatible polyurethane membrane matrix. Non-Patent Documents 1 to 6 disclose composite materials obtained by mixing polyurethane and silk fibroin.

### RELATED ART DOCUMENTS

### Patent Documents

Patent Document 1: Japanese Patent Application Publication No. 2017-052829
Patent Document 2: Japanese Patent Application Publication No. 2018-193624
Patent Document 3: Japanese Translation of PCT International Application Publication No. 2019-511339

### Non-Patent Documents

Non-Patent Document 1: Yasuhiro Fukuda et. al., “Relationship between structure and physical strength of silk fibroin nanofiber sheet depending on insolubilization treatment”, Journal of Applied Polymer Science, 2017, Vol. 134, No. 32, 45560
Non-Patent Document 2: Chikako T Nakazawa, et al., “Solid-state NMR studies for the development of non-woven biomaterials based on silk fibroin and polyurethane”, Polymer Journal, 2017, 49, p. 583-586
Non-Patent Document 3: Derya Aytemiz et al., “Compatibility Evaluation of Non-Woven Sheet Composite of Silk Fibroin and Polyurethane in the Wet State”, Polymers, 2018, Vol. 10, No. 8,874
Non-Patent Document 4: Kazumi Shimada et al., “The effect of a silk Fibroin/Polyurethane blend patch on rat Vessels”, Organogenesis, 2017, Vol. 13, No. 4, 115-124
Non-Patent Document 5: Chantawong P et al., “Effect of silk fibroin concentration on vascular remodeling in rat model”, Journal of Materials Science: Materials in Medicine, 2017, 28, 191
Non-Patent Document 6: Shimada, R. et al., “Development of a new surgical sheet containing both silk fibroin and thermoplastic polyurethane for cardiovascular surgery”, Surgery Today, 2017, 48, 486-494

### BRIEF DESCRIPTION OF THE DRAWINGS

FIG. 1 shows an SEM image of an appearance of Example 1.

FIG. 2 shows an SEM image of an appearance of Comparative Example 1.

FIG. 3 shows an SEM image of an appearance of Comparative Example 2.

FIG. 4 shows a result of an infiltrated tensile test of Example and Comparative Examples.

FIG. 5 shows a test result of the Young's modulus of Example and Comparative Examples.

FIG. 6 shows a test result of maximum stress of Example and Comparative Examples.

FIG. 7 shows a test result of yield stress of Example and Comparative Examples.

FIG. 8 shows a test result of breaking elongation of Example and Comparative Examples.

FIG. 9 shows a test result of yield elongation of Example and Comparative Examples.

### DESCRIPTION OF EXEMPLARY EMBODIMENTS

The present invention will be described below through embodiments of the invention, but the following embodiments do not limit the invention according to the claims. In addition, not all combinations of features described in the embodiments are essential to a solution of the invention. Note that in the drawings, the same or similar parts are assigned with same reference signs, and duplicated descriptions may be omitted.

### (I. Overview of Resin Composition)

According to the present embodiment, there is provided a resin composition that includes (A) a polymer compound (which may be referred to as a first polymer compound) including, in a main chain, a repeating unit (which may be referred to as a first repeating unit) formed of a plurality of amino acid residues bonded by peptide bond, and (B) a polymer compound (which may be referred to as a second polymer compound) including, in a terminal of a side chain, a peptide chain or a polypeptide chain formed of a plurality of amino acid residues bonded by peptide bond. The above-described resin composition is obtained, for example, by mixing the first polymer compound and the second polymer compound.

In the present embodiment, a monomer or a combination of a plurality of monomers that constitute the first polymer compound and a monomer or a combination of a plurality of monomers that constitute the second polymer compound are different. That is, the first polymer compound and the second polymer compound are different in type.

In recent years, it has been practiced to produce a polymer blend by mixing different types of polymers to develop a material with new properties and/or functionalities that are different from those of each polymer that constitutes the polymer blend. However, in general, different types of polymers are thermodynamically incompatible, and thus adhesiveness at an interface between the different types of polymers constituting the polymer blend is decreased. The decrease in the adhesiveness at the interface between the different types of polymers may affect the mechanical properties and/or degrading behavior of the polymer blend. Examples of degrading behavior include material degradation due to long-term use, delamination between different types of polymers, or the like.

The present inventors have conceived an idea to provide, to a terminal of the side chain of one polymer, a structure which relatively strongly interacts with a repeating unit of another polymer. The mutual solubility (which may be referred to as compatibility) between different types of polymers is improved by the local interaction at the interface between the different types of polymers. As a result, adhesiveness at the interface between the different types of polymers is improved, and thus the mechanical properties of the polymer blend are improved. Examples of mechanical properties include maximum stress, yield stress, breaking elongation, yield elongation, Young's modulus, or the like.

The present inventors have conceived of applying the above-described idea to a polymer blend of fibroin and other types of polymers. Fibroin has excellent biocompatibility. Thus, a fibroin-containing material is suitable for use in medical materials or scaffold materials for cell culture. In particular, the fibroin-containing material with the improved mechanical properties is particularly suitable for use in medical materials that require mechanical properties according to an intended application, in addition to cell affinity, low inflammation, and/or blood compatibility.

### (A. First Polymer Compound)

In the present embodiment, the first polymer compound includes, in its main chain, a repeating unit (which may be referred to as a first repeating unit, as described above) in which a plurality of amino acid residues are bonded. By the reaction of a plurality of amino acids, the repeating unit in which the plurality of amino acid residues, respectively derived from the plurality of amino acids, are bonded by peptide bond is obtained.

The above-described amino acid may be an α-amino acid in which the amino group and the carboxy group are bonded to the same carbon atom. At least some of the plurality of amino acids that constitute the first repeating unit may be α-amino acids. All of the amino acids that constitute the first repeating unit may be α-amino acids. Each of the plurality of amino acids described above may be an α-amino acid that is selected from a group consisting of glycine (Gly, or G), alanine (Ala, or A), valine (Val, or V), leucine (Leu, or L), isoleucine (Ile, or I), serine (Ser, or S), threonine (Thr, or T), aspartic acid (Asp, or D), glutamic acid (Glu, or E), lysine (Lys, or K), arginine (Arg, or R), phenylalanine (Phe, or F), tyrosine (Tyr, or Y), tryptophan (Trp, or W), histidine (His, or H), proline (Pro, or P), cysteine (Cys, or C), methionine (Met, or M), asparagine (Asn, or N), and glutamine (Gln, or Q), independently of one another.

The first polymer compound may be a polypeptide or a protein, or a derivative thereof, formed of the plurality of amino acid residues bonded by peptide bond. By the reaction of the plurality of amino acids, the polypeptide or the protein is obtained in which the plurality of amino acid residues respectively derived from the plurality of amino acids are bonded by peptide bond. The N-terminal amino acid and the C-terminal amino acid that constitute the polypeptide or the protein may be included in the amino acid residues described above.

The first polymer compound may be fibroin or a derivative thereof. When the first polymer compound is fibroin or a derivative thereof, the plurality of amino acid residues that constitute the described first repeating unit include amino acid residues derived from at least two types of amino acids selected from a group consisting of, for example, glycine, alanine, serine, tyrosine, and valine.

Fibroin includes a crystalline region and a semicrystalline region. The ratio of the crystalline region in fibroin is about 53 mol %, and the ratio of the semicrystalline region in fibroin is about 27 mol %. A repetitive amino acid sequence of the crystalline region in fibroin is represented as (GAGAGS)ₙ. Here, n is an integer from 1 to 20, for example. A repetitive amino acid sequence of the semicrystalline region in fibroin is represented as ((GX)ₛGY)ₜ. Here, X is A or V. In addition, s is an integer from 1 to 10, for example, and t is an integer from 1 to 20, for example.

In an embodiment, the plurality of amino acid residues that constitute the first repeating unit described above include amino acid residues derived from at least two types of amino acids selected from a group consisting of glycine, alanine, and serine. The amino acid sequence of the first repeating unit may be the same as the repetitive amino acid sequence, or a part thereof, of the crystalline region in fibroin.

As will be described below, the first polymer compound including fibroin is not limited to fibroin derived from natural silk. Thus, the first polymer compound can include a repetitive amino acid sequence that is different from the repetitive amino acid sequence included in fibroin derived from natural silk.

The amino acid sequence of the first repeating unit may be GA, AG, or AS. The amino acid sequence of the first repeating unit may be GAG, AGA, GGA, GAS, or any sequence including these. The amino acid sequence of the first repeating unit may be GAGA, AGAS, or any sequence including these. In these cases, a number of the first repeating unit included in the first polymer compound ranges from one or more to 30 or less, for example. A number of the first repeating unit included in the first polymer compound may range from one or more to 20 or less. A number of the first repeating unit included in the first polymer compound may range from two or more to 6 or less.

In another embodiment, the plurality of amino acid residues that constitute the first repeating unit described above include amino acid residues derived from at least two types of amino acids selected from a group consisting of glycine, alanine, tyrosine, and valine. The amino acid sequence of the first repeating unit may be the same as the repetitive amino acid sequence, or a part thereof, of the semicrystalline region in fibroin.

As described above, the first polymer compound including fibroin is not limited to fibroin derived from natural silk. Thus, the first polymer compound can include a repetitive amino acid sequence that is different from the repetitive amino acid sequence included in fibroin derived from natural silk.

The amino acid sequence of the first repeating unit may be GY, GA or AG, GV or VG, or any sequence including these. The amino acid sequence of the first repeating unit may be GGY, AGY, VGY, or any sequence including these. In these cases, a number of the first repeating unit included in the first polymer compound ranges from one or more to 30 or less, for example. A number of the first repeating unit included in the first polymer compound may range from one or more to 20 or less. A number of the first repeating unit included in the first polymer compound may range from two or more to 6 or less.

More specifically, the amino acid sequence of the first repeating unit may be GG or GA. In this case, the first repeating unit included in the first polymer compound ranges from one or more to 20 or less, for example. The first repeating units included in the first polymer compound may range from two or more to six or less.

### (Producing Method of Fibroin)

In an embodiment, fibroin may be silk fibroin derived from natural silk produced by silkworms or spiders. Fibroin is preferably silk fibroin derived from silk (which may be referred to as silkworm silk) produced by silkworms. In another embodiment, fibroin may be derived from silk protein produced by genetic engineering. Examples of silk proteins produced by genetic engineering can include silk proteins produced by bacteria, yeasts, animal and plant cells, transgenic plants, transgenic animals, or the like, which are genetically modified to produce silk proteins. The silk protein produced by genetic engineering includes, for example, a repetitive sequence of fibroin.

In silkworm silk, fibroin is coated with sericin. Fibroin, which is derived from natural silkworm silk, is obtained by removing sericin from silkworm silk. In an embodiment, the composition may contain, as an impurity, 10% to 35% by mass of sericin with respect to a mass of fibroin. In another embodiment, a content of sericin in the composition is, with respect to the mass of fibroin, preferably lower than 20% (mass ratio), more preferably lower than 10% (mass ratio), and further more preferably lower than 5% (mass ratio).

### (Fibroin Derivatives)

Examples of fibroin derivatives include compounds in which a part of fibroin is modified with any functional group. As described above, fibroin may be derived from natural silk, or may be derived from silk protein produced by genetic engineering.

### (B. Second Polymer Compound)

In the present embodiment, the second polymer compound includes, in a terminal of a side chain, a peptide chain or a polypeptide chain formed of a plurality of amino acid residues bonded by peptide bond. By the reaction of a plurality of amino acids, the peptide chain or the polypeptide chain is obtained in which the plurality of amino acid residues respectively derived from the plurality of amino acids are bonded. The N-terminal amino acid and the C-terminal amino acid that constitute the peptide chain or the polypeptide chain may be included in the amino acid residues described above.

### (Main Chain)

In the present embodiment, a monomer that constitutes the main chain of the second polymer compound is not limited to a particular monomer. The main chain of the second polymer compound may be constituted of a plurality of types of monomers.

### (Side Chain)

As described above, in the present embodiment, a peptide chain or a polypeptide chain formed of a plurality of amino acid residues bonded by peptide bond is attached to the terminal of the side chain of the second polymer compound. The plurality of amino acid residues that constitute the above-described peptide chain or polypeptide chain may include amino acid residues derived from at least two types of amino acids that constitute the first repeating unit described above.

Thus, when the resin composition is produced by mixing the first polymer compound and the second polymer compound, the compatibility between the first polymer compound and the second polymer compound is improved due to the local interaction occurring between the above-described peptide chain or polypeptide chain and the first repeating unit described above. As a result, the resin composition with excellent mechanical properties is obtained. As described above, examples of mechanical properties include maximum stress, yield stress, breaking elongation, yield elongation, Young's modulus, or the like.

As described above, in the present embodiment, the plurality of amino acid residues that constitute the peptide chain or the polypeptide chain include, for example, amino acid residues derived from at least two types of amino acids that constitute the first repeating unit. The amino acid sequence of the above-described peptide chain or polypeptide chain may be the same as a part of the amino acid sequence of the first repeating unit. The amino acid sequence of the above-described peptide chain or polypeptide chain may be the same as the amino acid sequence of the first repeating unit.

The above-described polypeptide chain includes, for example, a repeating unit (which may be referred to as a second repeating unit) formed of a plurality of amino acid residues bonded by peptide bond. By the reaction of a plurality of amino acids, the second repeating unit is obtained in which the plurality of amino acid residues respectively derived from the plurality of amino acids are bonded by peptide bond.

The plurality of amino acid residues that constitute the second repeating unit may include at least two types of amino acid residues that constitute the first repeating unit. The plurality of amino acid residues that constitute the second repeating unit may include at least three or more types of amino acid residues that constitute the first repeating unit. The amino acid sequence of the second repeating unit may be the same as a part of the amino acid sequence of the first repeating unit. The amino acid sequence of the second repeating unit may be the same as the amino acid sequence of the first repeating unit. The amino acid sequence of the second repeating unit may be determined, for example, so that a degree of hydrophobic interaction between the second repeating unit and at least part of the first repeating unit becomes greater than a predetermined degree.

A number of the second repeating unit included in the above-described polypeptide chain ranges from one or more to 50 or less, for example. A number of the second repeating unit included in the above-described polypeptide chain may range from one or more to 40 or less, or may range from one or more to 30 or less. A number of the second repeating unit included in the above-described polypeptide chain may range from two or more to 50 or less, may range from two or more to 40 or less, or may range from two or more to 30 or less, for example.

As described above, fibroin or a derivative thereof includes the repetitive amino acid sequence of (GAGAGS)n and ((GX)sGY)t. Here, n is an integer from 1 to 20, for example, s is an integer from 1 to 10, for example, and t is an integer from 1 to 20, for example. In addition, X is A or V. As such, when the first polymer compound is fibroin or a derivative thereof, any amino acid sequence exemplified as the amino acid sequences of the first repeating unit can be adopted as the amino acid sequence of the above-described peptide chain or polypeptide chain. When the first polymer compound is fibroin or a derivative thereof, the amino acid sequence of the above-described peptide chain or polypeptide chain may be (GA)u, (GAGAGS)n, or ((GX)sGY)t, or any amino acid sequence including these. Here, u is an integer from 1 to 10, for example.

### (Side Chain Structure)

In an embodiment, the second polymer compound may be the above-described peptide chain or polypeptide chain. For example, the second polymer compound in which the peptide chain or the polypeptide chain is attached to the side chain is obtained by the reaction between a carboxy group of a polymer compound (which may be referred to as a third polymer compound) in which at least one of a carboxy group or an amino group is introduced to the main chain, and an N-terminal amino group of the above-described peptide chain or polypeptide chain. Similarly, the second polymer compound in which the peptide chain or the polypeptide chain is attached to the side chain is obtained by the reaction between an amino group of the above-described third polymer compound and a C-terminal carboxy group of the above-described peptide chain or polypeptide chain.

In another embodiment, the side chain of the second polymer compound includes (i) the above-described peptide chain or polypeptide chain, and (ii) a linker. The above-described linker connects the main chain of the second polymer compound and the above-described peptide chain or polypeptide chain. In an embodiment, the N-terminal amino group of the peptide chain or the polypeptide chain and the linker are bonded. In another embodiment, the C-terminal carboxy group of the peptide chain or the polypeptide chain and the linker are bonded.

Examples of linkers include glycols, such as ethylene glycol, polyethylene glycol, or derivatives thereof. Other examples of linkers include polyglycolic acid, polycaprolactone, polylactic acid, or the like. The number of carbon atoms in the linker may range from two or more to 4000 or less, from two or more to 2000 or less, or from two or more to 1000 or less.

In still another embodiment, the second polymer compound includes a first side chain that includes, in a terminal thereof, the peptide chain or the polypeptide chain, and a second side chain that includes, in a terminal thereof, a functional peptide, an ester group, or a substituted or unsubstituted amide group. The first side chain and the second side chain bond to different carbon atoms in the main chain of the second polymer compound, for example.

Examples of functional peptides include REDV, IKVAV, YIGSR, RGD, SVVYGLR, or the like. Thus, adhesion ability, proliferation ability, migration property, and/or angiogenesis ability of a fibroblast cell, an endothelial cell, smooth muscle cell, or the like are improved.

The ester group and the amide group are introduced, for example, in order to seal an unmodified carboxy group that is introduced to the third polymer compound described above. The ester group is formed, for example, by the above-described carboxy group and alcohols, such as ethylene glycol or polyethylene glycol, bonding to each other. The amide group is formed, for example, by the above-described carboxy group and an amino acid bonding to each other.

### Specific Example of Second Polymer Compound

The second polymer compound includes at least one type of polymer compound which is selected from a group consisting of, for example, polyurethane, poly D lactic acid (PDLA), poly L lactic acid (PLLA), poly DL lactic acid (PDLLA), poly(ε-caprolactone) (PCL), polyglactin, polyethylene carbonate, polyglycolic acid (PGA), collagen, gelatin, chitosan, casein, keratin, sericin, polymethyl methacrylate (PMMA), polyamide, cellulose, polyhydroxyalkanoate (PHA), polybutylene succinate (PBS), glucose, polyvinyl alcohol (PVA), polyester, polyhydroxybutyrate (PHB), polymalic acid (PMA), poly(p-Dioxanone) (PDS, PDO), polypropylene glycol (PPG), poly(lactic acid-glycolic acid copolymer) (PLGA), polybutylene succinate (PBS), hydroxyapatite, poly(butylene adipate-co-terephthalate) (PBAT), polyethylene adipate-terephthalate (PEAT), tetra-polyethylene glycol (PTE), sodium polyacrylate (SAP), poly(sodium 4-styrenesulfonate) (PSS), poly(diallyldimethylammonium chloride) (PDDA), poly(2-methoxyethyl acrylate) (PMEA), polyacrylic acid (PAA), polyethyl acrylate (PEA), polybutyl acrylate (PBA), polyacrylamide (PAM), poly-methacrylate, poly(maleic anhydride), and poly(trimethylene carbonate) (PTMC), and derivatives thereof, and which includes in a terminal of a side chain thereof a peptide chain or a polypeptide chain formed of a plurality of amino acid residues bonded by peptide bond. Thus, the resin composition with excellent biocompatibility and mechanical properties is obtained. The resin composition with excellent biocompatibility and mechanical properties is suitable for use in medical materials or scaffold materials for cell culture.

The second polymer compound includes at least one type of polymer compound which is selected from a group consisting of, for example, polyurethane, polyurea, natural rubber, polycarbonate, and derivatives thereof, and which includes, in a terminal of a side chain, a peptide chain or a polypeptide chain formed of a plurality of amino acid residues bonded by peptide bond. The above-described polyurethane, polyurea, and/or polycarbonate may be a homopolymer, or a copolymer. Examples of polycarbonate include polyethylene carbonate. Thus, the resin composition with particularly excellent biocompatibility and mechanical properties is obtained. The resin composition with excellent biocompatibility and mechanical properties is suitable for use in medical materials or scaffold materials for cell culture.

### One Example of Another Embodiment

In the present embodiment, details of the resin composition have been described with reference to, as an example, the resin composition that includes two types of polymer compounds. However, the resin composition is not limited to the present embodiment. In another embodiment, the resin composition may include three or more types of polymer compounds. For example, the resin composition includes one or more types of first polymer compounds and two or more types of second polymer compounds. For example, the resin composition includes two or more types of first polymer compounds and one or more types of second polymer compounds.

### (II. Composition of the Resin Composition)

In the present embodiment, a mass ratio between the first polymer compound and the second polymer compound ranges from, for example, 1:99 to 99:1. The mass ratio between the first polymer compound and the second polymer compound may range from 20:80 to 80:20, or from 30:70 to 70:30. The mass ratio between the first polymer compound and the second polymer compound may range from 40:60 to 60:40, or from 45:55 to 55:45.

The physical properties of the resin composition approach the physical properties of the first polymer compound as the ratio of the mass of the first polymer compound to the mass of the second polymer compound increases. For example, when the first polymer compound is SF and the second compound is polyurethane (which may be referred to as PU), the maximum stress decreases, the yield stress decreases, the Young's modulus increases, the breaking elongation decreases, and the yield elongation decreases in the resin composition as the ratio of the mass of the first polymer compound to the mass of the second polymer compound increases.

Similarly, the physical properties of the resin composition approach the physical properties of the second polymer compound as the ratio of the mass of the first polymer compound to the mass of the second polymer compound decreases. For example, when the first polymer compound is SF and the second compound is polyurethane (which may be referred to as PU), the maximum stress increases, the yield stress increases, the Young's modulus decreases, the breaking elongation increases, and the yield elongation increases in the resin composition as the ratio of the mass of the first polymer compound to the mass of the second polymer compound decreases. As a result, an elastic region in the resin composition is enlarged. Enlarging the elastic region in the resin composition enables appropriate adjustment of the physical properties of the resin composition according to needs.

### (III. Physical Properties of the Resin Composition)

### (Young's Modulus)

In the present embodiment, the resin composition has a Young's modulus of, for example, 2 MPa or more and 20 MPa or less. The resin composition may have the Young's modulus of 2 MPa or more and 15 MPa or less. The Young's modulus of the resin composition is derived in compliance with, for example, ISO 527-1, JIS K 7161.

Specifically, first, a test piece to be measured is prepared. The shape and size of the test piece are those of JIS NO. 7 dumbbell shape, for example. In addition, a thickness of the test piece in a dry state is measured. The thickness of the test piece may be a thickness of one spot of the test piece, or may be an average value of thicknesses of a plurality of spots. Then, in an atmosphere of 20° C., a tensile load of 5 mm/min per minute is applied on the test piece, and tensile stress (which may be referred to as normal stress) and elongation (which may be referred to as strain, percent elongation, or the like) are measured while the test piece is pulled in its long side direction.

The tensile stress [MPa] is calculated by dividing the tensile load [N] by a cross-sectional area [mm²] of the test piece before the test starts. The above-described cross-sectional area is an area of a surface obtained by cutting the sample piece in a plane substantially perpendicular to the tensile direction. In addition, elongation [%] is calculated by the following Mathematical Expression 1.

$$Elongation [ \% ] = 100 \times ( L - Lo ) / Lo \qquad ( Math . 1 )$$

In Math. 1, Lo is a length of the sample before the test starts, and L is a length of the sample at the time of the test.

The Young's modulus is derived as a ratio of the tensile stress to the elongation within the tensile proportional limit (which may be referred to as an elastic range). In the present embodiment, the Young's modulus is derived by a gradient of a tangent line of the SS curve (which may be referred to as a stress-strain diagram). The gradient of the tangent line is derived by the data of the stress relative to the strain of 1% to 6%, for example.

Note that it is assumed that the calculation of the Young's modulus based on the above-described procedure is sometimes difficult due to the availability of samples, or the like. For example, in JIS K7161, a state of a test piece (which may be referred to as a sample) is adjusted according to specifications of a material to be tested. Unless otherwise specified regarding the adjustment of the state of the test piece, the state is recommended to be adjusted for 16 hours or more under conditions of a temperature of 21° C. to 25° C. and a humidity of 40% to 60%. However, depending on the material, physical properties may vary greatly between a wet state and a dry state. Examples of the above-described materials include (i) biopolymers such as collagen, fibrin, alginic acid, hyaluronic acid, fibroin (for example, silk fibroin), and sericin (for example, silk sericin), (ii) polyvinyl alcohol, polyglycolic acid, polyglactin, or the like. In such a case, the Young's modulus can be derived, for example, based on a tensile test in water (which may be referred to as an infiltrated tensile test) or a test that is equivalent to the tensile test in water, as described below.

### (Calculation Procedure of the Young's Modulus Based on Tensile Test in Water)

First, a resin composition is immersed in ultrapure water at 37° C. for 24 hours or more. Then, a water-containing test piece to be measured is cut out from the central region of the resin composition. The shape and size of the sample piece are those of JIS NO. 7 dumbbell shape, for example. Thus, the water-containing test piece is obtained.

Then, a thickness of the water-containing test piece is measured. The thickness of the test piece may be a thickness of one spot of the test piece, or may be an average value of thicknesses of a plurality of spots.

Then, a tensile test in water is performed using EZ Graph from Shimadzu Corporation. The tensile test in water is performed in water at 37° C., with a length between grips being 12 mm and at a tension rate of 5 mm/min. The number of times of measurement trials is set to 6 or more, for example. A stress [Pa] and a strain [%] are calculated based on a test load [N], a displacement [mm], a membrane thickness [mm], and a sample length [mm] obtained by the tensile test in water. A stress-strain curve (Stress-Strain curve) is produced by plotting a measurement result with the vertical axis representing the stress and the horizontal axis representing the strain. The Young's modulus is calculated based on a stress relative to a strain of 1 to 6%.

### (Maximum Stress)

In the present embodiment, a resin composition has a maximum stress of 1 MPa or more and 20 MPa or less, for example. The resin composition may have the maximum stress of 1 MPa or more and 15 MPa or less, or may have the maximum stress of 1 MPa or more and 10 MPa or less. The maximum stress of the resin composition can be adjusted by (i) a type of the first polymer compound and/or the second polymer compound, (ii) a content of, or a rate of modification to be described below of, the peptide chain or the polypeptide chain in the second polymer compound, and (iii) a mass ratio between the first polymer compound and the second polymer compound, or the like.

The maximum stress of the resin composition is derived in compliance with ISO 527-1, JIS K 7161, for example. The maximum stress of the resin composition is defined, for example, as a maximum value of the tensile stress described in connection with the deriving method of the Young's modulus. Specifically, a maximum value in the measured values of the tensile stress measured from the start of the above-described test until a time point at which the test piece breaks or at which the test reaches its maximum stroke is derived as the maximum stress of the resin composition.

### (Breaking Elongation)

In the present embodiment, the resin composition has a breaking elongation of, for example, 10% or more and 200% or less. The resin composition may have a breaking elongation of 10% or more and 150% or less. The breaking elongation of the resin composition can be adjusted by (i) a type of the first polymer compound and/or the second polymer compound, (ii) a content of, or a rate of modification to be described below of, the peptide chain or the polypeptide chain in the second polymer compound, and (iii) a mass ratio between the first polymer compound and the second polymer compound, or the like.

The breaking elongation of the resin composition is derived in compliance with ISO 527-1, JIS K 7161, for example. The breaking elongation of the resin composition is defined, for example, as a maximum value of the elongation described in connection with the deriving method of the Young's modulus. Specifically, the maximum value in the measured values of the elongation measured from the start of the above-described test until a time point at which the test piece breaks or at which the test reaches its maximum stroke is derived as the breaking elongation of the resin composition.

### (Yield Elongation)

In the present embodiment, the resin composition has a yield elongation of, for example, 5% or more and 100% or less. The resin composition may have a yield elongation of 5% or more and 60% or less. The yield elongation of the resin composition can be adjusted by (i) a type of the first polymer compound and/or the second polymer compound, (ii) a content of, or a rate of modification to be described below of, the peptide chain or the polypeptide chain in the second polymer compound, and (iii) a mass ratio between the first polymer compound and the second polymer compound, or the like.

The yield elongation of the resin composition is derived in compliance with ISO 527-1, JIS K 7161, for example. The yield elongation of the resin composition is defined, for example, as an elongation at a yield point. The yield point is defined, for example, as an intersection point between a tangent line having a gradient corresponding to the Young's modulus and a tangent line at a point where the gradient is parallelized immediately before breaking. Specifically, in the stress-strain curve described in connection with the deriving method of the Young's modulus, a position of the intersection point between the tangent line in the elastic deformation region of the curve and the tangent line in the plastic deformation region of the curve is calculated. Thus, the yield point is derived. In addition, the elongation at the above-described yield point is derived as the yield elongation.

### (Yield Stress)

In the present embodiment, the resin composition has a yield stress of, for example, 0.5 MPa or more and 10 MPa or less. The resin composition may have the yield stress of 0.5 MPa or more and 8 MPa or less. The yield stress of the resin composition can be adjusted by (i) a type of the first polymer compound and/or the second polymer compound, (ii) a content of, or a rate of modification to be described below of, the peptide chain or the polypeptide chain in the second polymer compound, and (iii) a mass ratio between the first polymer compound and the second polymer compound, or the like.

The yield stress of the resin composition is derived in compliance with ISO 527-1, JIS K 7161, for example. The yield stress of the resin composition is defined, for example, as a stress at the yield point described above. Specifically, in the stress-strain curve described in connection with the deriving method of the Young's modulus, a position of the intersection point between the tangent line in the elastic deformation region of the curve and the tangent line in the plastic deformation region of the curve is calculated. Thus, the yield point is derived. In addition, the stress at the above-described yield point is derived as the yield stress.

### (IV. Shape and Application of Resin Composition)

In the present embodiment, the shape of the resin composition is not limited to a particular shape. The resin composition has, for example, (i) a fibrous shape, (ii) a non-woven fabric shape, (iii) a sheet shape or a film shape, (iv) a tube shape or a roll shape, or (v) a block shape, a sponge shape, a pad shape, or a columnar shape.

In the present embodiment, the resin composition is used for applications to, for example, medical materials, scaffold materials for cell culture, functional apparel materials, or general industrial products made of silk. Examples of medical materials include an artificial blood vessel, a cardiac-repair patch, an artificial valve, a drug release material, an anti-adhesion material, an artificial bone, an artificial cartilage, a wound dressing, or the like.

In particular, devices intended for regeneration of blood vessels, hearts, or surrounding tissues thereof require mechanical properties suitable for the environments of the implantation sites, in addition to cell affinity, low inflammation, and blood compatibility. For example, according to the embodiment in which the first polymer compound is fibroin or a derivative thereof, the resin composition having (i) excellent biocompatibility and (ii) excellent Young's modulus, breaking elongation, maximum stress, yield stress, and/or yield elongation is obtained. The plastic deformation is suppressed in the resin composition according to the above-described embodiment, and thus, the resin composition is particularly suitable, for example, for the application to cardiovascular tissue engineering devices that function under dynamic environments. Examples of cardiovascular tissue engineering devices include an artificial blood vessel, a cardiac-repair patch, an artificial valve, or the like.

### (V. Production Method of Resin Composition)

In the present embodiment, the resin composition is produced by mixing the first polymer compound and the second polymer compound that are previously prepared. In the present embodiment, a procedure to prepare the first polymer compound is not limited to a particular procedure. The first polymer compound can be produced by a well-known production method for an intended compound.

In the present embodiment, although a procedure to prepare the second polymer compound is not limited to a particular procedure, the second polymer compound may be produced according to the following procedure, for example. First, the third polymer compound described above is prepared. As described above, the third polymer compound is a polymer compound which is different from the first polymer compound and the second polymer compound, and in which at least one of a carboxy group or an amino group is introduced to the main chain.

Examples of procedures to introduce at least one of a carboxy group or an amino group into the main chain of the polymer compound include a procedure to perform any well-known surface treatment on a polymer compound that includes a carbonate group. Examples of the above-described surface treatments include plasma irradiation treatment, chlorination treatment, plasma irradiation treatment, or the like.

Then, a peptide or a polypeptide that include at least two types of amino acid residues that constitute the first repeating unit is prepared. The peptide or the polypeptide can be produced by a well-known production method for an intended compound. Then, at least one of the carboxy group or the amino group in the third polymer compound is caused to react with the peptide or the polypeptide. Thus, the second polymer compound is obtained.

The ratio (which may be referred to as a rate of modification) of a number of mole of a functional group, modified with the peptide or the polypeptide, of either one of the carboxy group or the amino group of the third polymer compound relative to a number of mole of the functional group is, for example, 0.1 or more and 1 or less (or 10% or more and 100% or less). The rate of modification may be expressed in percentage. The rate of modification may be 0.1 or more and 0.5 or less (that is, 10% or more and 50% or less).

### (Measurement of the Rate of Modification)

The above-described rate of modification is derived, for example, by a BCA protein assay. The BCA protein assay is performed, for example, according to the following procedure. First, a dilution series of standard solution is produced. As the standard solution, bovine serum albumin (BCA) is used. As the dilution series, five series are produced which are 0 μg/mL, 100 μg/mL, 200 μg/mL, 400 μg/mL, and 800 μg/mL.

Then, a working solution is produced by mix a BCA Reagent A (main component: sodium bicinchoninate) and BCA Reagent B (main component: copper sulfate pentahydrate) so that BCA Reagent A:BCA Reagent B=100:1 (by volume) is obtained. Then, each of the five series of standard solutions and the above-described working solution are mixed to produce five series of mixed solutions. Each mixed solution is obtained by mixing 50 μl of the standard solution and 1 ml of the working solution. Subsequently, each mixed solution is bathed at 37° C. for 30 minutes.

Then, each of the five series of the dilution series described above is measured for light absorbance at 562 nm, using a ratio-beam spectrophotometer (U-5100 from Hitachi, Ltd.,). A measurement mode of the ratio-beam spectrophotometer is set to a quantitative operation. A number of standard test n is set to n=4. A calibration curve showing a relationship between the BSA concentration [μg/ml] and the light absorbance at 562 nm is created by using the above-described measurement results.

Then, samples for measurement are prepared. First, a solution (which may be referred to as a sample solution) of the second polymer compound to be measured is prepared. Then, after 50 μL of the sample solution and 1 mL of the working solution are mixed, the mixed solution is bathed at 37° C. for 30 minutes.

Then, the absorbance at 562 nm is measured by using the ratio-beam spectrophotometer (U-5100 from Hitachi, Ltd.,). A measurement mode of the ratio-beam spectrophotometer is set to a quantitative operation. A number of standard test n is set to n=4. The concentrations [μg/ml] of the peptide or the polypeptide in the sample solutions are derived by substituting the above-described measurement results into the calibration curve described above. Then, the number of mole MA [mol] of the peptide or the polypeptide in the sample solutions are calculated by using the concentrations of the peptide or the polypeptide in the sample solutions, amounts of sample solutions, dilution factors of the sample solutions, molecular weight of the peptide or the polypeptide, or the like.

On the other hand, the number of mole MB of the carboxy group or the amino group exposed on the main chain of the polymer compound (which is the third polymer compound described above), i.e., the polymer compound before the second polymer compound is modified with the peptide or the polypeptide, is presented by a manufacturer or a vendor of the third polymer compound, for example. The rate of modification is calculated by dividing the above-described MA by the above-described MB, and the obtained value is multiplied by 100.

### (Resin Composition of Fibrous Shape or Non-Woven Fabric Shape)

According to one embodiment, first, a solution (which may be referred to as a spinning dope) of a mixture of the first polymer compound and the second polymer compound is prepared. A solvent of the spinning dope may be an aqueous solvent or an organic solvent. Then, a fiber or a non-woven fabric is produced by using a spinning apparatus. For example, a filament of the resin composition is produced by the spinning dope being discharged from a nozzle of the spinning apparatus. The non-woven fabric of the resin composition is produced by accumulation of the above-described filament. In the above-described procedure, any well-known production method of the fiber or the non-woven fabric, such as an electrospinning method, a solution-blow method, or a melt-blown method can be adopted.

### (Resin Composition of Film Shape, Tube Shape, or Block Shape)

According to another embodiment, first, the first polymer compound and the second polymer compound are dissolved in an appropriate solvent to produce a mixture solution of the first polymer compound and the second polymer compound. Then, the above-described mixture solution is poured into an appropriate container or a mold. Subsequently, the solvent in the mixture solution is evaporated. The evaporation of the solvent may be performed at ambient temperature, at ultralow temperature (that is, freeze-dried), or at high temperature. The evaporation of the solvent may be performed at ambient pressure or at low pressure. Thus, the resin composition having (i) a sheet shape or a film shape, (ii) a tube shape or a roll shape, or (iii) a block shape, a sponge shape, a pad shape, or a columnar shape is produced.

### EXAMPLES

Hereinafter, the present invention will be described more specifically with reference to Example and Reference Examples. It should be noted that the present invention is not limited to the following Example unless the present invention goes beyond the gist.

### Example 1

First, 250 g of raw silk reeled from domesticated silkworm cocoons was immersed in an aqueous solution of 0.02M sodium carbonate (a special grade chemical from Wako Pure Chemical Industries, Ltd.) at 95° C., and stirred for 30 minutes to be scoured. Then, the scoured raw silk was cleaned 3 times with purified water at 40° C. Thus, sericin remaining in the scoured raw silk was almost completely removed. Subsequently, the fibers from which sericin had been removed were further cleaned with purified water, and then dried. Thus, a silk fibroin (which may be abbreviated as SF) fiber was obtained.

Then, the above-described SF was added to an aqueous solution of 9M lithium bromide (from Wako Pure Chemical Industries, Ltd.), and the SF was dissolved under the shaking conditions of 37° C. and 100 rpm. Thus, the SF solution was obtained. Subsequently, the lithium bromide was removed from the SF solution by dialysis treatment. In the dialysis treatment, a dialysis cell tube boiled for 20 minutes was used. The dialysis treatment was performed under the condition of 4° C., and water was exchanged three times a day. The dialysis treatment was ended when an electric conductivity of the purified water became 2 μS/cm or less after 10 hours or more has passed after the water exchange.

Then, impurities were removed from the dialysis-treated SF aqueous solution by centrifugation. The centrifugation was carried out for 30 minutes under conditions of 4° C. and 8500 rpm. In addition, an operation of removing the impurities by the centrifugation was performed twice in total. Subsequently, a small amount of the SF aqueous solution from which the impurities had been removed was added dropwise to a plurality of petri dishes, and the weight after drying was measured to measure a concentration.

Then, a concentration of the SF in the SF aqueous solution from which the impurities had been removed was prepared to be 1% (w/v) by using pure water. The SF aqueous solution having the prepared concentration was transferred to an eggplant flask, pre-frozen in liquid nitrogen, and then freeze-dried. Thus, an SF sponge was obtained.

### Synthesis Example 2

Two hundred milligrams of water-dispersible polyurethane (which may be abbreviated as WPU) having carboxy groups in the molecular chain and 1 mg of pure water were mixed. Thus, 20% (w/v) of WPU aqueous dispersion solution was obtained. The WPU aqueous dispersion solution having a prepared concentration was transferred to an eggplant flask, pre-frozen in liquid nitrogen, and then freeze-dried. Thus, a WPU sponge was obtained.

The WPU was added in a silicone mold, pre-frozen for three days in a −20° C. environment, and then the pre-frozen WPU was placed in a sample vial, again pre-frozen in liquid nitrogen, and then freeze-dried. Thus, WPU sponge was obtained.

### Synthesis Example 3

Two hundred milligrams of water-dispersible polyurethane (which may be abbreviated as WPU) having carboxy groups in the molecular chain and 1 mg of pure water were mixed. Thus, 20% (w/v) of WPU aqueous dispersion solution was obtained.

Then, a part of the carboxy groups in the WPU was modified with a peptide (H₂N-Gly-Ala-Gly-Ala-Gly-Ala-COOH) by EDC/NHS method. The peptide having the above-described amino group sequence of GAGAGA may be referred to as a model peptide.

Specifically, first, EDC/NHS solution was produced by mixing 7.668 mg of hydrochloride of 1-ethyl-3-(3-dimethylaminopropyl) carbodiimide (EDC), 21.714 mg of sodium salt of N-hydroxysuccinimide (NHS), and 18 mL of MES buffer. The MES buffer was produced by using 2-(morpholino) ethanesulfonic acid aqueous solution and sodium hydroxide aqueous solution, and the EDC/NHS solution was stirred for 30 minutes while a pH thereof was adjusted to be 5.4 by adding the produced MES buffer. EDC/NHS solution was adjusted at room temperature (23° C.).

Then, 32 mg of GA peptide was added to the EDC/NHS solution, and 12 mL of sodium hydroxide was added to adjust pH of the EDC/NHS solution to 7.4. By stirring this solution for 24 hours at room temperature, a solution (which may be referred to as a reaction solution) was obtained that includes polymers (which may be abbreviated as WPU-G) in which a part of the carboxy groups of the water-dispersible polyurethane was modified with model peptides, and unreacted model peptides.

Then, the unreacted model peptides were removed from the reaction solution by dialysis treatment using a dialysis membrane (from Japan medical science company) of which a molecular weight cut-off is 3500. The dialysis treatment was performed under the condition of 4° C., water was exchanged three times a day, and then the dialysis treatment was ended after a lapse of three days.

Then, the WPU-G aqueous solution was transferred to an eggplant flask, pre-frozen in liquid nitrogen, and then freeze-dried. Thus, WPU-G sponge was obtained.

### Example 1

In 5000 μl of 1,1,1,3,3,3-hexafluoro-2-propanol (HFIP) (from Sigma-Aldrich), 175 mg of the SF sponge obtained in the synthesis example 1 and 175 mg of the WPU-G sponge obtained in the synthesis example 3 were added, and then stirred for 15 hours under the condition of 300 rpm at room temperature. Thus, an HFIP solution of SF/WPU-G was obtained. SF: WPU-G (mass ratio) was 1:1. A concentration of the SF in the HFIP solution was 3.5% (w/v). A concentration of the WPU-G in the HFIP solution was 3.5% (w/v).

Then, a non-woven sheet of SF/WPU-G was produced by using the HFIP solution of

SF/WPU-G as a spinning dope and an electrospinning apparatus (Fluidnatek LE-50, from Bioinicia). An applied voltage of the electrospinning apparatus was set to 28 kv. A discharge amount of the spinning dope was set to 1.3 mL/h. A discharge distance was set to 15 cm.

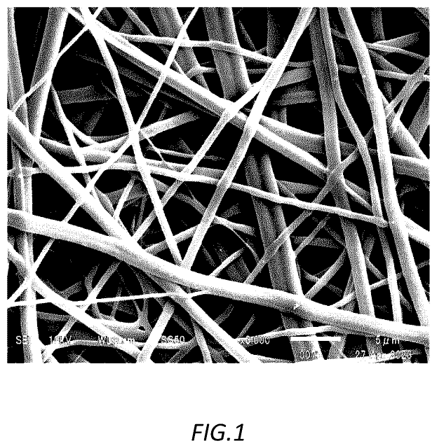

Then, an insolubilization treatment was performed on the silk fibroin in the non-woven fabric. Specifically, the produced non-woven sheet was, together with a collecting plate of the electrospinning apparatus, left to stand for two hours under the condition of a relative humidity of 100% and at a temperature of 37° C. Then, the insolubilized non-woven sheet was immersed in water together with the collecting plate, and the non-woven sheet was peeled from the collecting plate. Thus, the non-woven sheet of SF/WPU-G was obtained. An SEM image of the produced non-woven sheet is shown in FIG. 1.

### Comparative Example 1

In 5000 μL of 1,1,1,3,3,3-hexafluoro-2-propanol (HFIP) (from Sigma-Aldrich), 250 mg of the SF sponge obtained in the synthesis example 1 was added, and then stirred for 15 hours under the condition of 300 rpm at room temperature. Thus, an HFIP solution of SF was obtained. A concentration of the SF in the HFIP solution was 5% (w/v).

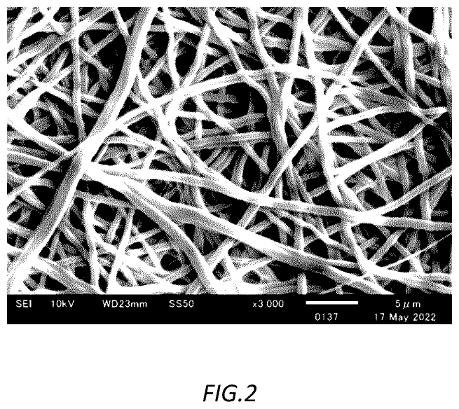

Subsequently, a non-woven sheet of SF was produced according to a procedure similar to that of Example 1. An applied voltage of the electrospinning apparatus was set to 25 kv. A discharge amount of the spinning dope was set to 1.3 mL/h. A discharge distance was set to 15 cm. An SEM image of the produced non-woven sheet of SF is shown in FIG. 2.

### Comparative Example 2

In 5000 μl of 1,1,1,3,3,3-hexafluoro-2-propanol (HFIP) (from Sigma-Aldrich), 175 mg of the SF sponge obtained in the synthesis example 1 and 175 mg of the WPU sponge obtained in the synthesis example 2 were added, and then stirred for 15 hours under the condition of 300 rpm at room temperature. Thus, an HFIP solution of SF/WPU was obtained. SF: WPU (mass ratio) was 1:1. A concentration of the SF in the HFIP solution was 3.5% (w/v). A concentration of the WPU in the HFIP solution was 3.5% (w/v).

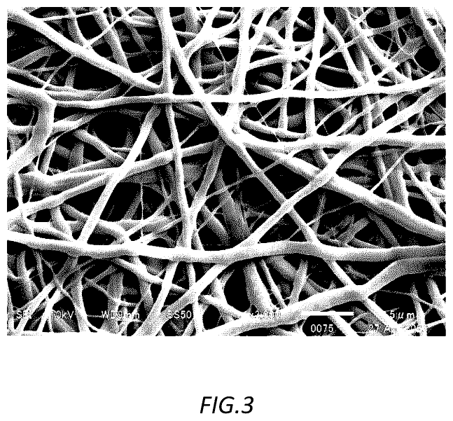

Subsequently, a non-woven sheet of SF/WPU was produced according to a procedure similar to that of Example 1. An applied voltage of the electrospinning apparatus was set to 28 kv. A discharge amount of the spinning dope was set to 1.3 mL/h. A discharge distance was set to 15 cm. An SEM image of the produced non-woven sheet of SF/WPU is shown in FIG. 3.

### (Evaluation)

### (Evaluation of Mechanical Property)

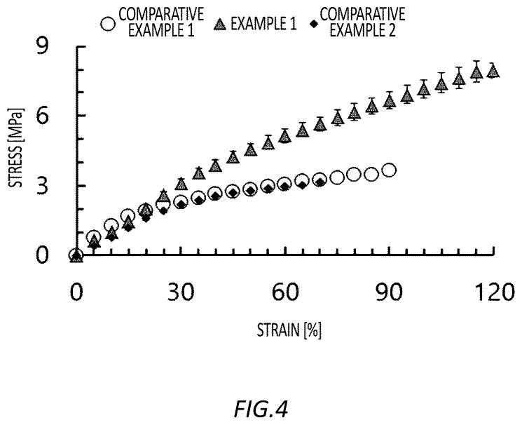

The tensile test in water described above was performed on the non-woven fabric obtained in each of Example 1, Comparative Example 1, and Comparative Example 2. The result of the tensile test in water on each of Example 1, Comparative Example 1, and Comparative Example 2 is shown in FIG. 4. FIG. 4 shows an SS curve obtained from the tensile test in water on each of Example 1, Comparative Example 1, and Comparative Example 2.

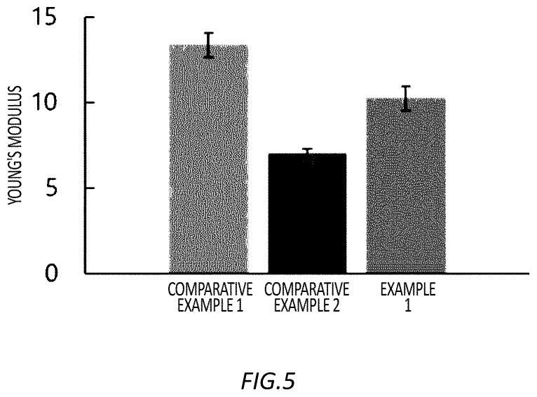

Based on the result of the tensile test in water, the Young's modulus of each of Example and Comparative Examples was calculated. The Young's modulus of the non-woven sheet of Example 1 was 10.2 [MPa]. The Young's moduli of the non-woven sheets of Comparative Example 1 and Comparative Example 2 were 13.3 [MPa] and 6.97 [MPa], respectively. The measurement result of the Young's modulus of the non-woven sheet of each of Example 1, Comparative Example 1, and Comparative Example 2 is shown in FIG. 5.

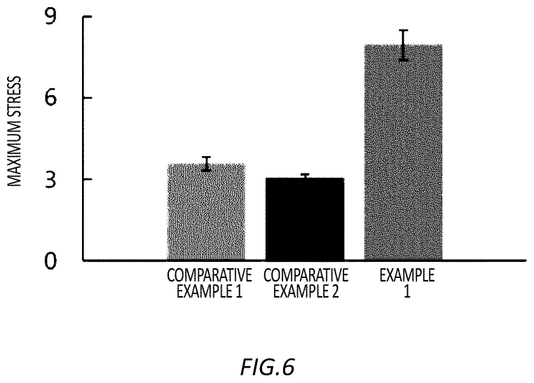

According to the procedure described above, the maximum stress was calculated for the non-woven fabric obtained in each of Example 1, Comparative Example 1, and Comparative Example 2. The maximum stress of the non-woven sheet of Example 1 was 7.95 [MPa]. The maximum stresses of the non-woven sheets of Comparative Example 1 and Comparative Example 2 were 3.58 [MPa] and 3.05 [MPa], respectively. The measurement result of the maximum stress of the non-woven sheet of each of Example 1, Comparative Example 1, and Comparative Example 2 is shown in FIG. 6.

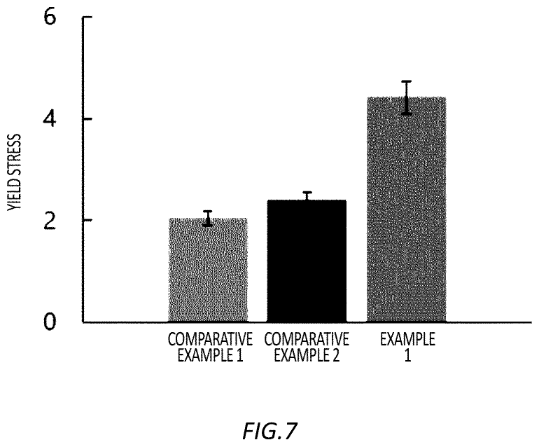

According to the procedure described above, the yield stress was calculated for the non-woven fabric obtained in each of Example 1, Comparative Example 1, and Comparative Example 2. The yield stress of the non-woven sheet of Example 1 was 4.41 [MPa]. The yield stresses of the non-woven sheets of Comparative Example 1 and Comparative Example 2 were 2.05 [MPa] and 2.4 [MPa], respectively. The measurement result of the yield stress of the non-woven sheet of each of Example 1, Comparative Example 1, and Comparative Example 2 is shown in FIG. 7.

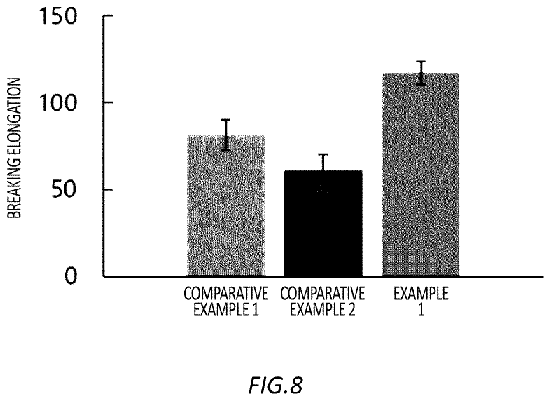

According to the procedure described above, the breaking elongation was calculated for the non-woven fabric obtained in each of Example 1, Comparative Example 1, and Comparative Example 2. The breaking elongation of the non-woven sheet of Example 1 was 117 [%]. The breaking elongations of the non-woven sheets of Comparative Example 1 and Comparative Example 2 were 81.2 [%] and 60.5 [%], respectively. The measurement result of the breaking elongation of the non-woven sheet of each of Example 1, Comparative Example 1, and Comparative Example 2 is shown in FIG. 8.

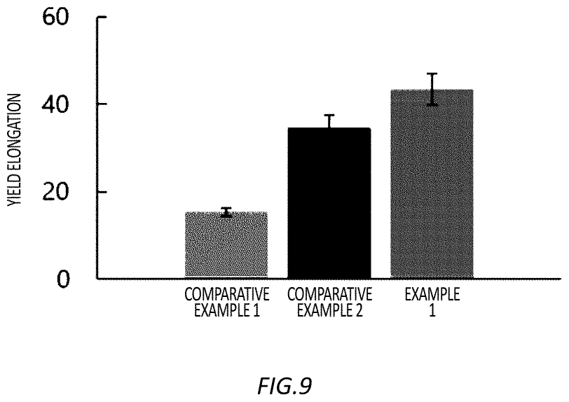

According to the procedure described above, the yield elongation was calculated for the non-woven fabric obtained in each of Example 1, Comparative Example 1, and Comparative Example 2. The yield elongation of the non-woven sheet of Example 1 was 43.4 [%]. The yield elongations of the non-woven sheets of Comparative Example 1 and Comparative Example 2 were 15.4 [%] and 34.5 [%], respectively. The measurement result of the yield elongation of the non-woven sheet of each of Example 1, Comparative Example 1, and Comparative Example 2 is shown in FIG. 9.

In FIGS. 5 to 9, when alphabetical letters shown on the respective graphs are different from each other, it indicates that there is a significant difference. As shown in FIGS. 5 to 9, it can be seen that the non-woven sheet of Example 1 has each of the mechanical properties that have been significantly improved compared to the non-woven sheets of Comparative Example 1 and Comparative Example 2.

### (Evaluation of Chemical Properties)

### (Rate of Modification)

The rate of modification was calculated for the non-woven fabric obtained in Example 1 according to the procedure described above. The rate of modification of WPU-G in the synthesis example 3 was 19.7%. That is, 19.7% of COOH groups in the WPU used to produce the WPU-G had been modified with the model peptide.

### (Compatibility)

A dynamic viscoelastic measurement (DMA) was performed on the non-woven fabric obtained in each of Example 1 and Comparative Example 2, and a glass transition temperature Tg [° C.] of the WPU and a glass transition temperature Tg [° C.] and a crystal relaxation temperature Tm [° C.] of the SF included in each of the non-woven fabrics were measured.

For the dynamic viscoelastic measurement, DVA 205 from IT Keisoku Seigyo Co., LTD was used. The temperature range was set to 100 to 300° C. The rate of temperature increase was set to 2° C./min. The frequency was set to 1 Hz. The length between grips was 25 mm. The number of times of the test was set to 3 or more. A storage elastic modulus, a loss elastic modulus, and a loss tangent (tan δ) of each sample were measured by using DVA 205. The above-described Tg and Tm were calculated from the peak top temperature of the above-described loss tangent.

The Tg of the WPU in the SF/WPU-G of Example 1 was −53.6±3.22° C. The Tg of the SF was 203±1.32°. In addition, the Tm of the SF was 284±1.29° C.

The Tg of the WPU in the SF/WPU of Comparative Example 2 was-56.9±3.53° C. The Tg of the SF was 204±1.00°. In addition, the Tm of the SF was 272±0.19° C.

According to the above-described measurement results, it can be seen that a difference in Tg between the WPU and the SF in the SF/WPU-G of Example 1 is smaller than a difference in Tg between the WPU and the SF in the SF/WPU of Comparative Example 2. The smaller the difference in Tg between different types of polymers, the more the compatibility between the different types of polymers improves. Accordingly, it can be seen that the compatibility between the SF and the PU has been improved by a part of the side chain in the WPU being modified with the model peptide.

In addition, according to the above-described measurement results, the Tm of the SF in the SF/WPU-G of Example 1 is higher than the Tm of the SF in the SF/WPU of Comparative Example 2 by 10° C. or more. That is, it can be seen that the thermal stability of the resin composition has been improved by the interaction between the crystalline region in the SF and the model peptide of the PU.

While the present invention has been described by way of the embodiments, the technical scope of the present invention is not limited to the above-described embodiments. It is apparent to persons skilled in the art that various alterations or improvements can be made to the above-described embodiments. In addition, the matters described with regard to the particular embodiment can be applied to other embodiments with a range without causing technical contradictions. It is also apparent from the described scope of the claims that the embodiments added with such alterations or improvements can be included the technical scope of the present invention.

It should be noted that the materials, and processes of operations, procedures, steps, stages, or the like in the production method of the materials shown in the claims, specification, and drawings can be performed in any order as long as the order is not indicated by “prior to,” “before,” or the like and as long as the output from a previous process is not used in a later process. Even if the process flow is described using phrases such as “first” or “then” in the claims, specification, or drawings, it does not necessarily mean that the process must be performed in this order.

What is claimed is:

1. A resin composition comprising:
a first polymer compound that has, in a main chain, a first repeating unit formed of a plurality of amino acid residues bonded by peptide bond; and
a second polymer compound that has, in a terminal of a side chain, a peptide chain or a polypeptide chain formed of a plurality of amino acid residues bonded by peptide bond, wherein
the plurality of amino acid residues that constitute the first repeating unit of the first polymer compound include amino acid residues derived from at least two types of amino acids selected from a group consisting of glycine, alanine, serine, tyrosine, and valine,
the first polymer compound is fibroin or a derivative thereof,
the peptide chain or the polypeptide chain of the second polymer compound includes a second repeating unit formed of a plurality of amino acid residues bonded by peptide bond, and an amino acid sequence of the second repeating unit is the same as an amino acid sequence, or a part thereof, of the first repeating unit, and
the amino acid sequence of the first repeating unit of the first polymer compound is GA, AG, GAG, AGA, or GAGA, or any amino acid sequence including any of these.

2. The resin composition according to claim 1, wherein
a number of the first repeating unit included in the first polymer compound ranges from one or more to 30 or less.

3. The resin composition according to claim 1, wherein
the amino acid sequence of the peptide chain or the polypeptide chain of the second polymer compound is (GA) u, (GAGAGS) n, or ((GX) sGY) t, or any amino acid sequence including any of these amino acid sequences, wherein u and s each indicate an integer from 1 to 10, n and t each indicate an integer from 1 to 20, and X indicates A.

4. The resin composition according to claim 1, wherein
a number of the second repeating unit included in the polypeptide chain ranges from one or more to 50 or less.

5. The resin composition according to claim 1, wherein
the side chain of the second polymer compound includes:
the peptide chain or the polypeptide chain, and
a linker that connects the peptide chain or the polypeptide chain and a main chain of the second polymer compound.

6. The resin composition according to claim 1, wherein
the second polymer compound includes:
a first side chain that includes, in a terminal thereof, the peptide chain or the polypeptide chain, and
a second side chain that includes, in a terminal thereof, a functional peptide, an ester group, or a substituted or unsubstituted amide group.

7. The resin composition according to claim 1, wherein
a mass ratio between the first polymer compound and the second polymer compound ranges from 1:99 to 99:1.

8. The resin composition according to claim 1, wherein
the second polymer compound has at least one type of polymer compound which is selected from a group consisting of polyurethane, poly D lactic acid (PDLA), poly L lactic acid (PLLA), poly DL lactic acid (PDLLA), poly(ε-caprolactone) (PCL), polyglactin, polyethylene carbonate, polyglycolic acid (PGA), collagen, gelatin, chitosan, casein, keratin, sericin, polymethyl methacrylate (PMMA), polyamide, cellulose, polyhydroxyalkanoate (PHA), polybutylene succinate (PBS), glucose, polyvinyl alcohol (PVA), polyester, polyhydroxybutyrate (PHB), polymalic acid (PMA), poly(p-Dioxanone) (PDS, PDO), polypropylene glycol (PPG), poly(lactic acid-glycolic acid copolymer) (PLGA), polybutylene succinate (PBS), hydroxyapatite, poly(butylene adipate-co-terephthalate) (PBAT), polyethylene adipate-terephthalate (PEAT), tetra-polyethylene glycol (PTE), sodium polyacrylate (SAP), poly(sodium 4-styrenesulfonate) (PSS), poly(diallyldimethylammonium chloride) (PDDA), poly(2-methoxyethyl acrylate) (PMEA), polyacrylic acid (PAA), polyethyl acrylate (PEA), polybutyl acrylate (PBA), polyacrylamide (PAM), poly-methacrylate, poly(maleic anhydride), and poly(trimethylene carbonate) (PTMC), and derivatives thereof, and which includes, in a terminal of a side chain thereof, a peptide chain or a polypeptide chain formed of a plurality of amino acid residues bonded by peptide bond.

9. The resin composition according to claim 1, wherein
the resin composition has (i) a fibrous shape, (ii) a non-woven fabric shape, (iii) a sheet shape or a film shape, (iv) a tube shape or a roll shape, or (v) a block shape, a sponge shape, a pad shape, or a columnar shape.

10. A medical material including a resin composition according to claim 1.

11. A medical material including a resin composition according to claim 2.

12. A medical material including a resin composition according to claim 3.

13. A medical material including a resin composition according to claim 4.

14. A medical material including a resin composition according to claim 5.

15. A medical material including a resin composition according to claim 6.

16. A medical material including a resin composition according to claim 7.

17. The medical material according to claim 10, wherein
the medical material is an artificial blood vessel, a cardiac-repair patch, an artificial valve, a drug release material, an anti-adhesion material, an artificial bone, an artificial cartilage, or a wound dressing.

18. A method for producing a resin composition, comprising:
preparing a first polymer compound that has, in a main chain, a first repeating unit formed of a plurality of amino acid residues bonded by peptide bond;
preparing a second polymer compound that has, in a terminal of a side chain, a peptide chain or a polypeptide chain formed of a plurality of amino acid residues bonded by peptide bond; and
mixing the first polymer compound and the second polymer compound to produce a resin composition that includes the first polymer compound and the second polymer compound, wherein
the plurality of amino acid residues that constitute the first repeating unit of the first polymer compound include amino acid residues derived from at least two types of amino acids selected from a group consisting of glycine, alanine, serine, tyrosine, and valine,
the first polymer compound is fibroin or a derivative thereof,
the peptide chain or the polypeptide chain of the second polymer compound includes a second repeating unit formed of a plurality of amino acid residues bonded by peptide bond, and an amino acid sequence of the second repeating unit is the same as an amino acid sequence, or a part thereof, of the first repeating unit, and
the amino acid sequence of the first repeating unit of the first polymer compound is GA, AG, GAG, AGA, or GAGA, or any amino acid sequence including any of these.

19. The method according to claim 18, wherein
the preparing the second polymer compound includes:
preparing a third polymer compound which is different from the first polymer compound and the second polymer compound, and in which at least one of a carboxy group or an amino group is introduced to a main chain;
preparing a peptide or a polypeptide that includes the second repeating unit; and
causing the at least one of the carboxy group or the amino group of the third polymer compound to react with the peptide or the polypeptide to produce the second polymer compound.

20. The method according to claim 19, wherein
a ratio of a number of mole of a functional group of either one of the carboxy group or the amino group of the third polymer compound which has been modified with the peptide or the polypeptide relative to a number of mole of the functional group of the either one ranges from 0.1 or more to 1 or less.
